(19) United States
(12) Patent Application Publication
(10) Pub. No.: US 2004/0087994 A1
(43) Pub. Date: May 6, 2004

(54) Mechanical bone tamping device for repair of osteoporotic bone fractures

(76) Inventor: Loubert Suddaby, Orchard Park, NY (US)

Correspondence Address: SHOEMAKER AND MATTARE, LTD, P.O. BOX 2286, 2001 JEFFERSON DAVIS HIGHWAY, ARLINGTON, VA, 22202-0286, US

(21) Appl. No.: 10/230,256

(22) Filed: Aug. 29, 2002

(51) Int. Cl. A61B 17/00

(52) U.S. Cl. 606/190

(57) ABSTRACT

A mechanical bone tamping device for osteoporotic repair include a pair of arms mounted on a spreading mechanism such as a screw jack. The mechanism is introduced into a small hole in a vertebra through a cannula, and is then operated to spread the arms apart, forming a cavity which may be filled with cement to fortify the vertebra.

### BACKGROUND OF THE INVENTION

Pathologic fracture of the spinal vertebral body is very common. Bones weakened by osteoporosis or by malignant processes account for a large proportion of vertebral fractures. Most such fractures occur as a result of trivial trauma and are due to the weakened architecture of the bone through loss of bone calcium and associated alteration of bony trabecular support or through frank replacement of bony tissue by malignant cells.

The injection of bone cement into the vertebral body to strengthen or stabilize it is a well recognized process that provides immediate stability to the weakened or compressed vertebral body that has been altered by disease. Present systems designed to inject bone cement into the vertebra weakened by disease (malignant or benign) generally utilize two types of processes. The first process involves simply injecting liquid bone cement into the interstices of the bone under pressure. The problem with this process is that it requires the bone cement to be in a relatively liquid state to allow it to fill the interstices of the bone. Because venous channels within the bone communicate with epidural veins in the spinal canal and with veins in the general vasculature, numerous complications have arisen from this injection process whereby bone cement has inadvertently entered the spinal canal causing paralysis from compressing the spinal cord or, alternately, cement has entered the general venous system, causing death by pulmonary embolism. Obviously, these consequences of injecting bone cement under pressure into the interstices or trabeculae of vertebral bodies are unacceptable.

A second and safer method has been developed to strengthen osteoporotic or malignant vertebral fractures. This involves placing a balloon into the intervertebral body and inflating it so that a cavity is formed in the weakened bone. This cavity can then be filled with a more viscous form of bone cement, thereby reducing the risk of embolism to the spinal canal or lungs as is seen with high pressure less viscous injection. The problem with this technique is that the balloons used to create the cavity within the bone frequently break when spicules of bone puncture them, or, because they expand along the path of least resistance, an aberrant or asymmetrical cavity is formed which inhibits or compromises the ideal placement of the cement support for stabilization of the weakened vertebrae. A more desirable system is required to allow placement of bone cement in the exact position required by the treating surgeon and in a manner that acceptably lessens the risk of bone cement migration or embolization.

### SUMMARY OF THE INVENTION

The system described herein is a simple mechanical mechanism whereby a cavity can be created in any desirable location within the vertebral body to allow the instillation of bone cement in a viscous configuration thereby minimizing the risk of malplacement of the bone cement or embolization of bone cement through the trabecular channels as may happen when less viscous bone cement is administered to strengthen pathologic cancellous bone.

To achieve this greater safety and efficacy, a mechanical device for creating a cavity within the soft cancellous bone is used. This form of cavity creation is much more controllable than with balloon inflation insofar as it does not depend on the elastic properties of a balloon wall expanding along the path of least resistance to create a cavity, whereas the dimensions of a balloon-created cavity are largely beyond the surgeon's control and more or less dependent upon the extent of disruption of the architecture of the pathologic bone.

According to this invention, a cavity is formed by compressing cancellous trabeculae outward, much as one might form a cavity in moist snow by inserting a hand, fingers extended, and then closing it to form a fist. To produce the cavity by purely mechanical action, a screw jack or other expanding mechanism is employed to compress or tamp the surrounding weakened cancellous bone. The mechanism, when operated, forces the arms apart, thereby directly compressing or tamping the cancellous bone.

By employing a screw jack mechanism to form the cavity, the exact dimensions of the cavity as well as the placement of the cavity can be controlled by the treating surgeon. Passive placement of liquid bone cement by injection under pressure is not required and the highly inaccurate and uncontrollable cavity formation afforded by balloon insufflation is avoided. The screw jack mechanism affords a more direct, exquisitely controllable and safer means by which cavities can be formed for bone cement stabilization of vertebrae weakened or fractured by benign or malignant disease states. Although a screw jack mechanism is envisioned in the preferred embodiment, it is recognized that other mechanisms such as levers could be substituted to achieve the same result, i.e., mechanical compression of cancellous bone to formulate a cavity within the confines of the vertebral body.

The important point of this invention is that the expanding device is purely mechanical, as opposed to balloon-type devices which have both mechanical and pneumatic aspects.

### BRIEF DESCRIPTION OF THE DRAWINGS

In the accompanying drawings,

FIG. 3 is an axial view of vertebral body with stylet inserted via posterolateral approach;

FIG. 4 shows a cannula sleeve inserted over the stylet;

FIG. 5 illustrates the working cannula in position with the stylet removed;

FIG. 6 demonstrates the screw jack being placed into the vertebral body via the working cannula;

FIG. 7 shows the screw jack in an open configuration thereby compressing bone adjacent to the expandable arms;

FIG. 8 shows the cavity formed after the screw jack has been repeatedly expanded and contracted at the 15 degree intervals;

FIG. 9 demonstrates the cavity being filled with cement after the screw jack is removed;

FIG. 10 depicts the bone cement in situ after the working cannula is removed;

FIGS. 11-18 are lateral views corresponding to FIGS. 3-10; and

FIGS. 19 and 20 show a modified form of the tamping instrument, using a lever mechanism.

### DESCRIPTION OF THE PREFERRED EMBODIMENT(S)

FIGS. 1 and two show devices designed to create a cavity within the bony contents of a vertebral body to allow or facilitate the stabilization of said vertebral body by instillation of bone cement or other stabilizing material (biological or inert) to repair, splint or otherwise stabilize bone structures weakened by benign or malignant processes (osteoporosis or malignant infiltration).

The screw jack tamp or lever arm bone compression instrument shown in FIG. 1 includes a shaft 10 having a handle (not shown) at one end to allow mechanical rotation of the shaft and a radially expandable structure 14 at the other end having two or more pressure arms 16, each of which extends along an axis parallel to that of the shaft. Each arm 16 is supported at its midpoint by a pair of links 18 having pivot pin connections at either end: one end to the arm, and one end to either of two collars mounted some distance apart on the shaft. The distal collar 20 is held at one end of the shaft against a shoulder (not shown) by a retainer such as a snap ring 28. There is some free play, so the distal collar can rotate with respect to the shaft, but it cannot move axially. The proximal collar 22 has an internal screw thread which mates with an external thread 30 extending over a portion of the length of the shaft near the distal end.

The thread shown is left-handed, so that clockwise rotation of the shaft advances the proximal collar 22 toward the distal collar 20. This approximation of the collars forces the pivot arms outward, so that the pressure arms compress the surrounding soft cancellous bone. The device is contracted by rotating the shaft handle counter clockwise, and then is reexpanded after the entire assembly has been rotated slightly. By repeating this process, an approximately cylindrical cavity is eventually formed.

An alternative form of the invention is shown in FIG. 2. Here, a sleeve of strong, inelastic fabric mesh 32 is placed around the pressure arms so that, when the arms are extended, the mesh compresses soft bone between the arms as well. This modification potentially reduces the number of times the device must be incrementally rotated and reexpanded.

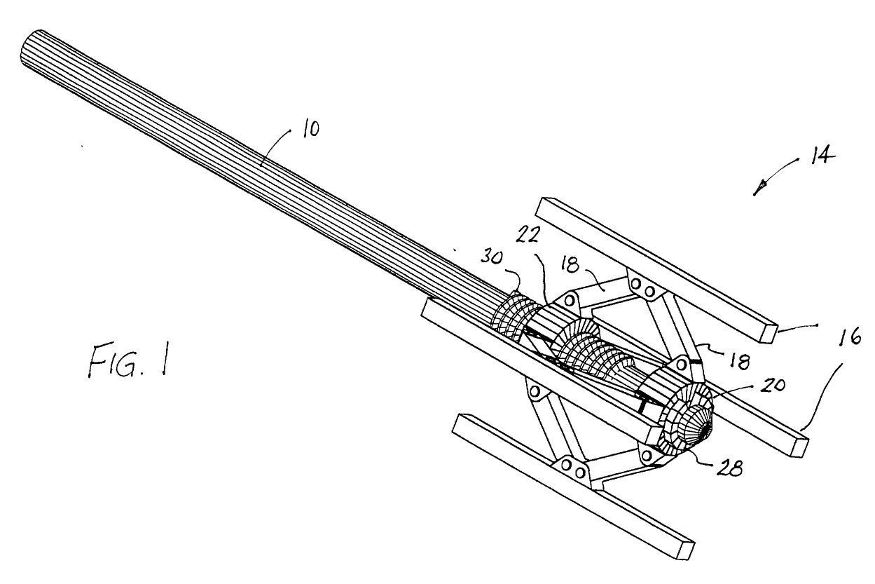

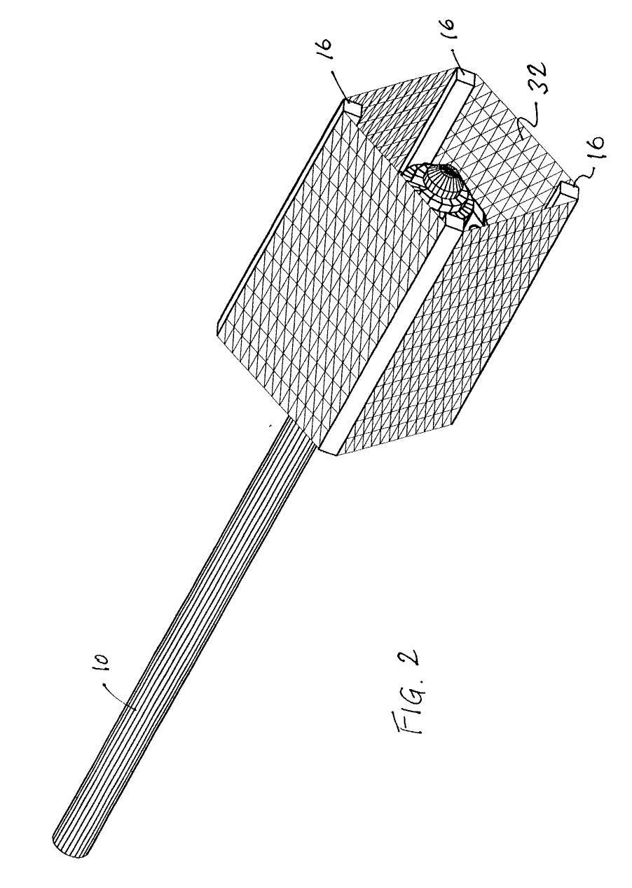

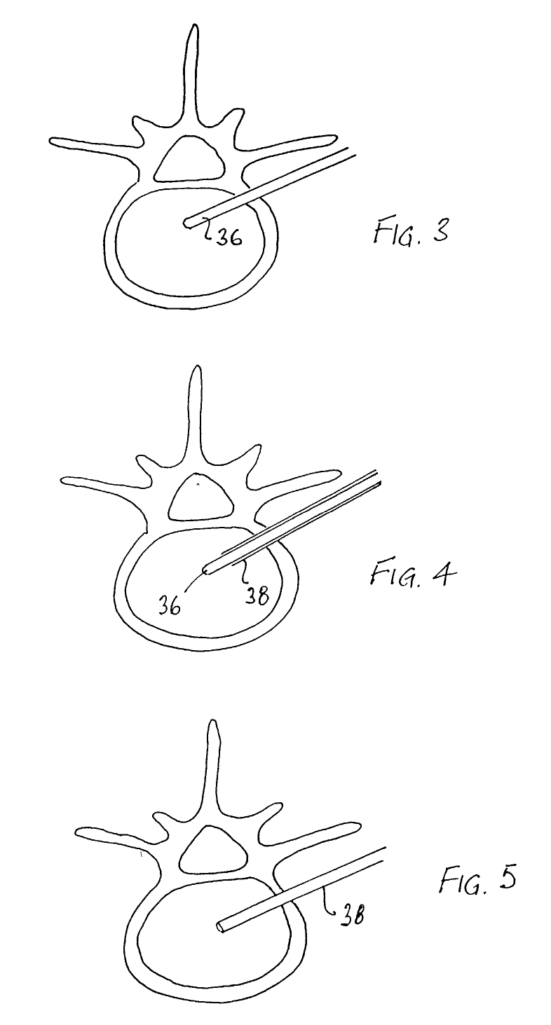

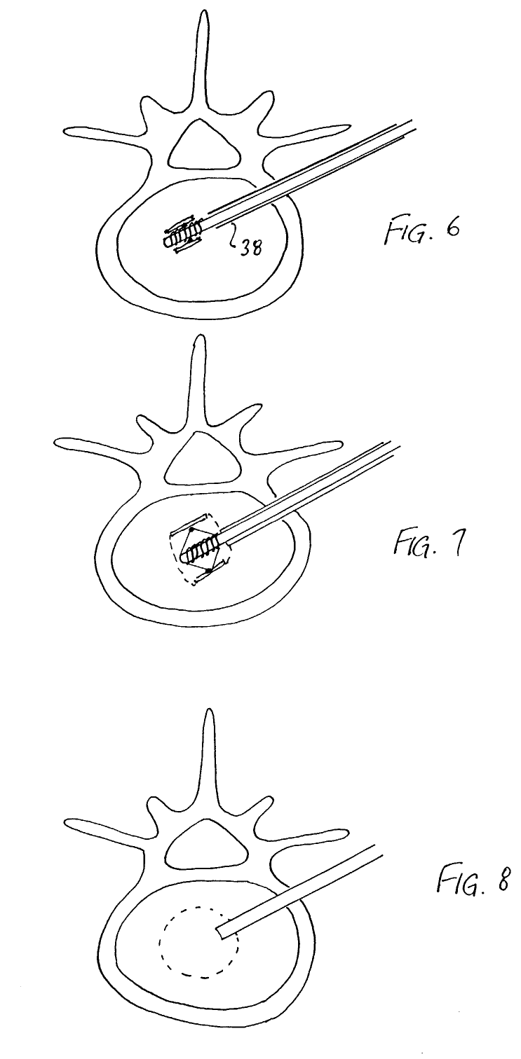

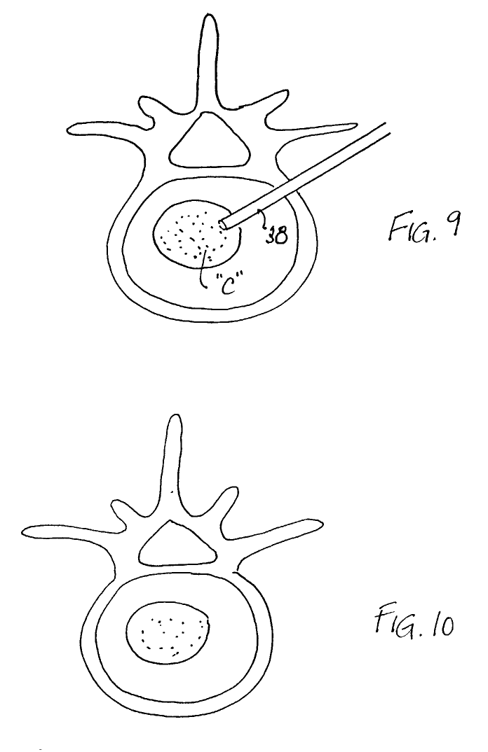

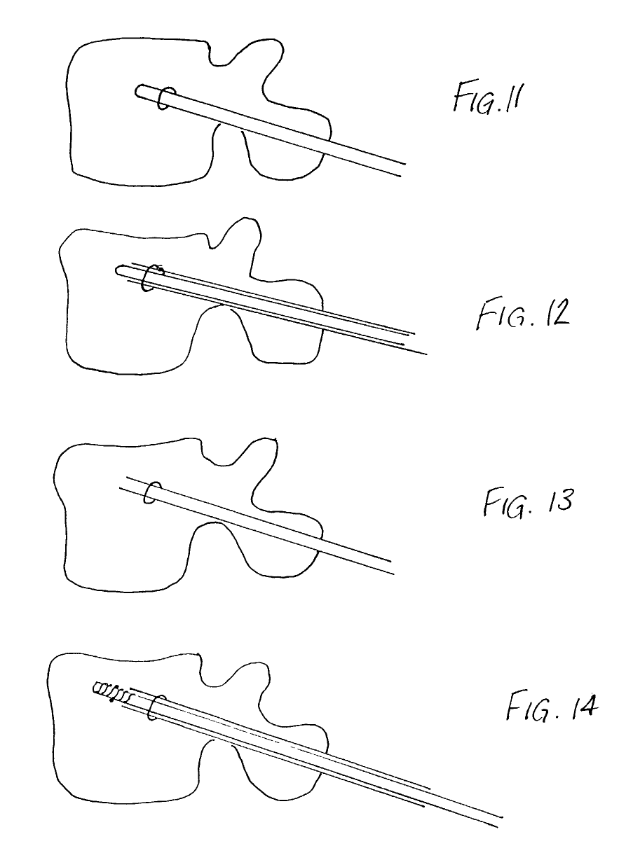

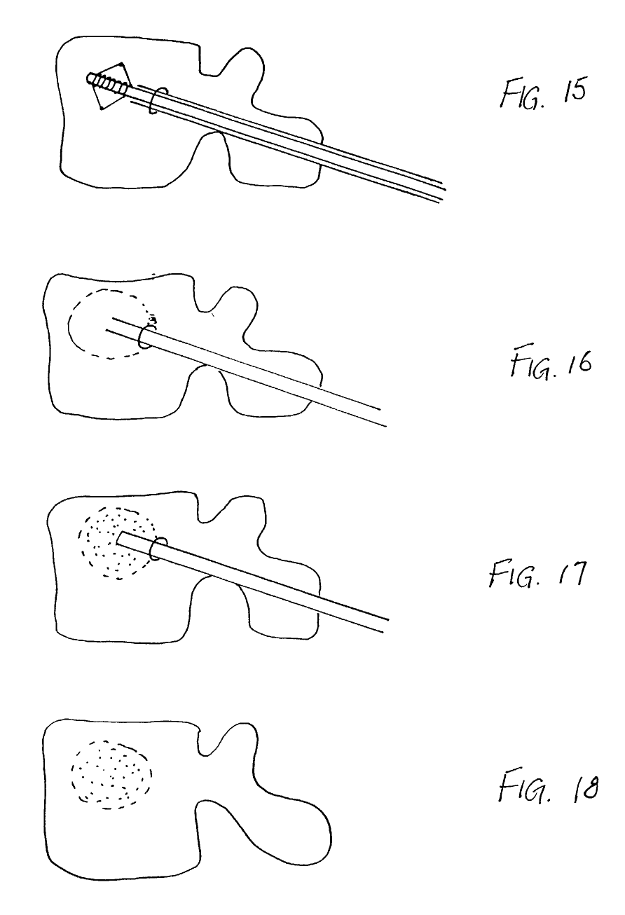

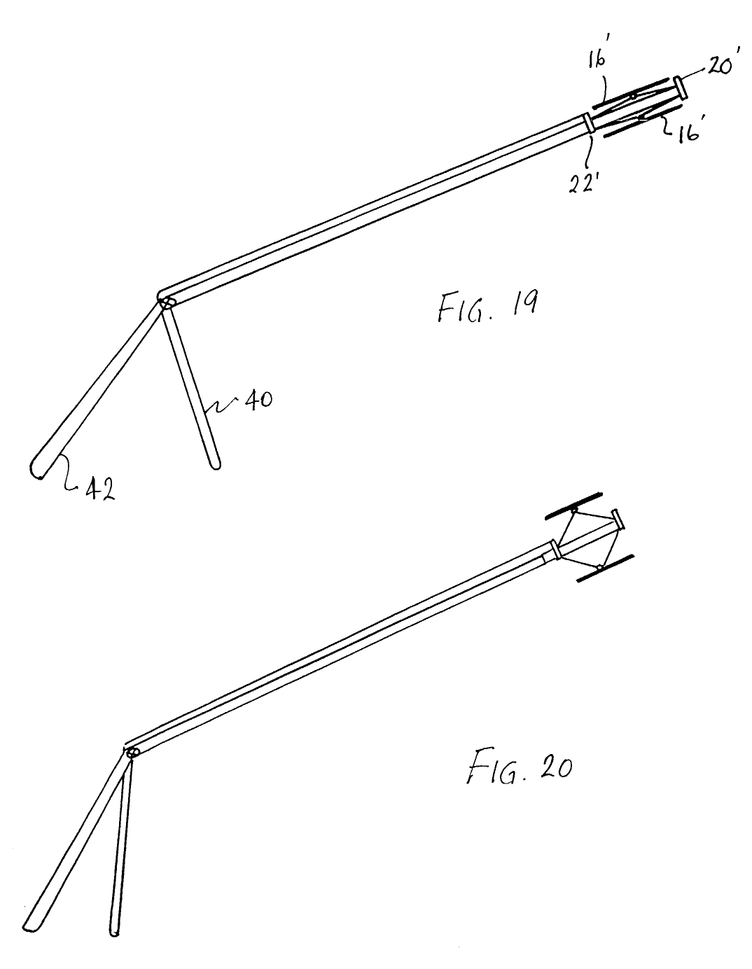

In operation, a blind hole is formed in the vertebra by inserting a stylet 36 (FIG. 3). A cannula sleeve 38 is then inserted over the stylet (FIG. 4), and the stylet is removed (FIG. 5). Now the surgeon inserts the tool described above (FIG. 6), and then turns its handle (not shown) clockwise to expand the arms (FIG. 7), enlarging the cavity in the plane of the arms. The arms are then retracted, and the screw jack is turned somewhat (e.g., 15°-45°—the exact angular interval required will depend on the desired size of the cavity and the width of the arms) and then the arms are expanded again. This cycle repeated as many times necessary to cover 360° and produce a cavity which is substantially round in cross-section (FIG. 8). Bone cement in a more or less viscous state is now injected along the cannula to fill the cavity (FIG. 9). The cement is allowed to harden in the cavity to stabilize the weakened or fractured osteoporotic bone. Finally, the cannula is withdrawn and the hole closed (FIG. 10). FIGS. 11-18 are lateral views corresponding to FIGS. 3-10.

FIGS. 19 and 20 show a form of the invention in which the arms are expanded not by a screw jack, but rather by a lever-based tool, in which squeezing the handles 40, 42 together shortens the distance between the collars 20′,22′, thus expanding the arms 16. The effect and method of operation is the same, although the mechanical advantage may not be as great.

Since the invention is subject to modifications and variations, it is intended that the foregoing description and the accompanying drawings shall be interpreted as only illustrative of the invention defined by the following claims.

I claim:

1. A mechanical bone tamping device for forming cavities in soft cancellous bone, said device comprising
at least two elongate arms, and
a mechanical spreading mechanism connected to each of the arms, for spreading the arms apart,
said mechanism and said arms being adapted to be passed, when the arms are not spread apart, through a cannula into a hole formed in the bone.

2. The invention of claim 1, wherein said mechanism comprises a screw jack having
a shaft with a threaded portion,
a stationary collar supported on the shaft, and
a movable collar having internal threads engaged with said threaded portion,
each of said arms being supported on said collars by a first link having a pivot connection to said stationary collar and a second link having a pivot connection to said movable collar.

3. The invention of claim 1, wherein said mechanism comprises
a forceps having a pair of handles an elongate body portion,
a stationary collar supported on the body,
a movable collar mounted for sliding movement along the body,
means connecting one of said handles to said traveling collar in such a way that squeezing the handles together draws the collars toward one another,
each of said arms being supported on said collars by a first link having a pivot connection to said stationary collar and a second link having a pivot connection to said movable collar.

4. A method of forming a cavity in soft cancellous bone, said method comprising steps of
forming a hole in said bone,
introducing a cannula into the hole,
inserting a mechanically expandable tool into the hole through the cannula, and
expanding the tool in the hole to form an enlarged cavity within the bone.

5. A method of stabilizing a bone weakened by osteoporosis, said method comprising steps of
forming a hole in said bone,
introducing a cannula into the hole,
inserting a mechanically expandable tool into the hole through the cannula,
expanding the tool in the hole to form an enlarged cavity within the bone,
collapsing the tool,
withdrawing the tool through the cannula,
injecting bone cement through the cannula so as to fill the cavity, and
allowing the cement to harden.
